(19) United States
(12) Patent Application Publication
(10) Pub. No.: US 2022/0293283 A1
(43) Pub. Date: Sep. 15, 2022

(54) Systems and Methods for Medical Concept Mapping

(71) Applicant: Intelligent Medical Objects, Inc., Rosemont, IL (US)

(72) Inventors: Sahas Subramanian, Fox River Grove, IL (US); Andrew S. Kanter, Highland Park, IL (US); Greg Aldin, Oak Park, IL (US); Rajiv Haravu, Hawthorn Woods, IL (US); Jay Jayakumar, Prospect Heights, IL (US); Matt Cardwell, Oak Park, IL (US)

(21) Appl. No.: 17/200,446

(22) Filed: Mar. 12, 2021

(51) Int. Cl. G16H 70/00; G16H 15/00; G16H 30/40

(52) U.S. Cl. CPC G16H 70/00; G16H 15/00; G16H 30/40

(57) ABSTRACT

A medical concept code analysis system includes a medical terminology source and at least one processor configured to access the medical terminology source. The processor also is configured to receive interface terminology concept code information associated with a plurality of interface terminology concept codes, receive public concept code information associated with a plurality of public concept codes, generate annotations for the plurality of public concept codes based on the public concept code information by determining a string and/or substring of the description associated with each public concept code contains an annotation that align with one or more predetermined annotation categories, associate the annotations with the plurality of interface terminology concept codes, and cause the interface terminology concept codes and the annotations to be output.

### CROSS-REFERENCE TO RELATED APPLICATIONS

N/A.

### STATEMENT REGARDING FEDERALLY SPONSORED RESEARCH

N/A.

### BACKGROUND OF THE INVENTION

Many medical concept terminologies are used by various medical institutions (e.g., hospitals, research facilities, etc.) in classifying patient diseases, disorders, and/or other medical conditions. For example, SNOMED CT (SCT) codes can be used by a number of medical organizations in generating patient reports. As another example, International Code of Diseases (ICD) codes can be used in billing and/or reimbursement processes to classify specific patient conditions. Specifically, ICD-9 and/or ICD-10 are commonly used by hospitals and/or other care centers in billing insurance providers.

Using the correct and/or most relevant concept code is important for several reasons. One reason is that the correct code may improve the accuracy of bills generated for a patient. For example, in a bill generated for childbirth, there may be several different grades of difficulty in childbirth that can be selected and/or other codes indicating that non-standard work was performed (e.g., a Caesarean section was performed). Thus, certain terminologies such as ICD-10 may include more codes related to childbirth than simply “childbirth.” Certain codes may result in greater reimbursement for the doctor and/or hospital that perform a certain procedure.

While ICD codes are commonly used for purposes of reimbursement, doctors (e.g., oncologists, surgeons, cardiologists, etc.) may prefer using other terminologies. For example, some doctors may use SNOMED CT codes in describing patient symptoms and/or conditions (e.g., in a patient report) because it offers more descriptive concepts (i.e., more granular concepts) than other codes, such as ICD-9 and/or ICD-10. Additionally, some SNOMED CT codes may not be specific enough to accurately align with an equivalent ICD code. Some doctors may write a patient report using non-standardized terminology (e.g., not included in the SNOMED CT concepts) and/or too broad of terminology that does not immediately align with ICD codes. In order to receive reimbursement, medical organizations may need trained medical staff to “translate” the patient reports into ICD codes that can be used in a billing report, which can increase the time a doctor may spend on generating billing reports and/or increase the cost to the patient and/or insurance company.

Additionally, medical practitioners may desire to research possible conditions of a patient when the patient's condition is unclear. Current systems may not provide sufficient granularity in describing conditions and/or symptoms, which can lead to inefficient lookup of conditions and/or treatment of the patient.

It would therefore be desirable to provide systems and methods that improve the lookup of standardized medical terms (e.g., concepts, codes, etc.) and/or patient conditions and/or symptoms, as well as automatically understand the clinical importance and/or intent of medically related statements without losing the meaning of the statements

### SUMMARY OF THE INVENTION

The present disclosure provides systems and methods that overcome one or more of the aforementioned drawbacks by providing new systems and methods for lookup of medical terms.

In accordance with one aspect of the disclosure, a medical concept code analysis system is provided. The system includes a medical terminology source and at least one processor configured to access the medical terminology source and configured to receive interface terminology concept code information associated with a plurality of interface terminology concept codes, receive public concept code information associated with a plurality of public concept codes, generate annotations for the plurality of public concept codes based on the public concept code information by determining a string and/or substring of the description associated with each public concept code contains an annotation that align with one or more predetermined annotation categories, associate the annotations with the plurality of interface terminology concept codes, and cause the interface terminology concept codes and the annotations to be output.

In accordance with another aspect of the disclosure, a medical concept code analysis system is provided. The system includes a medical terminology source and at least one processor configured to access the medical terminology source and configured to receive public concept code information associated with a plurality of public concept codes arranged in a hierarchy, flatten the hierarchy, receive interface terminology concept code information associated with a plurality of interface terminology concept codes, each of the plurality of interface terminology concept codes being associated with at least one of the plurality of public concept codes, receive inclusion criteria and exclusion criteria, generate a valueset of interface terminology concept codes based on the inclusion criteria and exclusion criteria, and cause the valueset to be output.

The foregoing and other aspects and advantages will appear from the following description. In the description, reference is made to the accompanying drawings which form a part hereof, and in which there is shown by way of illustration configurations of the invention. Any such configuration does not necessarily represent the full scope of the invention, however, and reference is made therefore to the claims and herein for interpreting the scope of the invention.

### BRIEF DESCRIPTION OF THE DRAWINGS

FIG. 1 is an example of an image analysis system in accordance with the disclosed subject matter.

FIG. 2 is an example of hardware that can be used to implement a computing device and a supplemental computing device shown in FIG. 1 in accordance with the disclosed subject matter.

FIG. 3 is an example of a simple interface terminology concept mapping.

FIG. 4 is an example of an improved interface terminology concept mapping.

FIG. 5 is an exemplary interface terminology concept code mapping.

FIG. 6 is an exemplary ontological graph of an interface terminology concept code.

FIG. 7 is an exemplary process for generating annotations for interface terminology concept codes based on public concept codes.

FIG. 8 is an exemplary graphical user interface (GUI) for searching a number of interface terminology concept codes.

FIG. 9 is an exemplary process for generating valuesets representing hierarchical relationships between concepts.

FIG. 10A is an exemplary GUI for searching a patient database using a number of interface terminology concept codes.

FIG. 10B is another exemplary GUI for searching a patient database using a number of interface terminology concept codes.

FIG. 10C is yet another exemplary GUI for searching a patient database using a number of interface terminology concept codes.

### DETAILED DESCRIPTION

The present disclosure provides systems and methods that can streamline and/or enhance the lookup of standardized medical terms (e.g., concepts, codes, etc.) and/or patient conditions and/or symptoms, thereby providing an optimized or enhanced user interface or user experience.

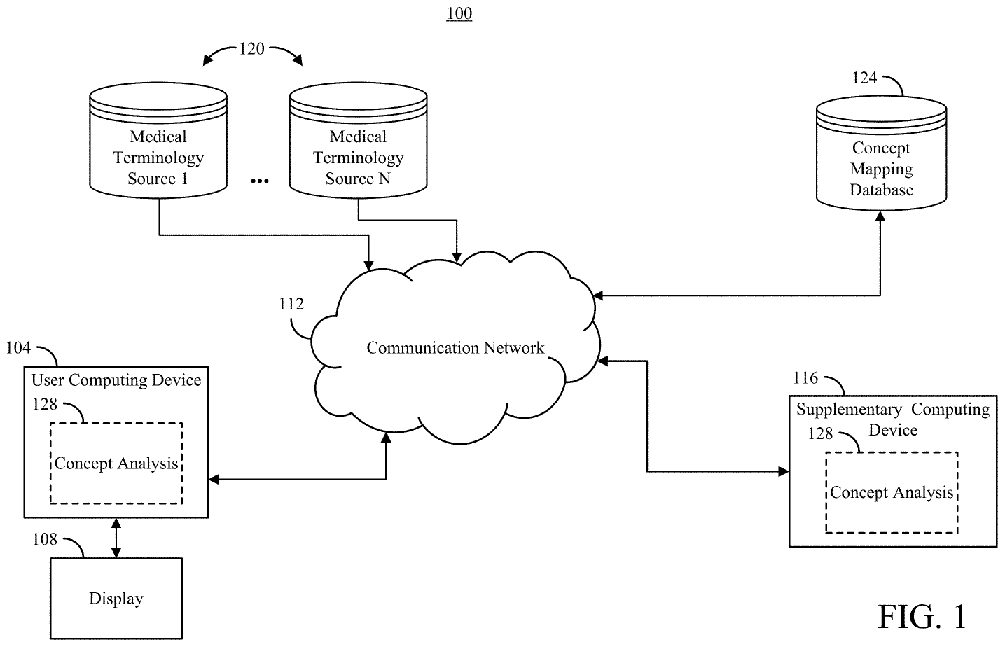

FIG. 1 shows an example of a concept analysis system 100 in accordance with some aspects of the disclosed subject matter. In some embodiments, the concept analysis system 100 can include a computing device 104, a display 108, a communication network 112, a secondary or supplemental computing device 116, one or more medical terminology sources 120, and a concept mapping database 124. The computing device 104 can be in communication (e.g., wired communication, wireless communication) with the display 108, the supplemental computing device 116, the medical terminology source(s) 120, and the concept mapping database 124.

The computing device 104 can implement portions of a concept analysis application 128, which can involve the computing device 104 transmitting and/or receiving instructions, data, commands, etc., from one or more other devices. For example, the computing device 104 can receive medical terminology data (e.g., medical concept codes such as ICD codes, SNOMED CT codes, etc.) from the medical terminology source(s) 120, receive and/or transmit concept data from the concept mapping database 124, and/or transmit reports and/or search results generated by the concept analysis application 128 to the display 108.

The supplemental computing device 116 can implement portions of the concept analysis application 128. It is understood that the concept analysis system 100 can implement the concept analysis application 128 without the supplemental computing device 116. In some aspects, the computing device 104 can cause the supplemental computing device 116 to receive medical terminology data from medical terminology source(s) 120, receive and/or transmit mapped concept codes from the concept mapping database 124, and/or transmit reports and/or search results generated by the concept analysis application 128 to the display 108. In this way, a majority of the concept analysis application 128 can be implemented by the supplemental computing device 116, which can allow a larger range of devices to be used as the computing device 104 because the required processing power of the computing device 104 may be reduced.

The medical terminology source(s) 120 can include standardized medical concept codes such as ICD codes, SNOMED CT concept codes, RXNorm concept codes, CPT4 concept codes, and/or other suitable standardized medical concept codes. The medical terminology source(s) 120 can also include metadata associated with the medical concept codes, such as hierarchy information indicating any parents and/or children of the medical concept codes.

The concept mapping database 124 can include a collection of interface terminology concept codes, each of which can be mapped to one or more publicly available standardized medical concept codes (e.g., ICD codes, SNOMED CT concept codes, RXNorm concept codes, CPT4 concept codes, etc.). In one aspect, the interface terminology concept codes may be codes within an interface terminology such as the interface terminology structured in the commonly-assigned U.S. Pat. No. 9,594,872 and/or U.S. Patent Publication No. 2012/0179696, the contents of each which are incorporated herein by reference in their entirety. Additionally, as will be described below, the interface terminology concept codes can be mapped to a hierarchy generated based on hierarchy information associated with the standardized medical concept codes.

The concept analysis application 128 can provide a search function to a user to browse the collection of interface terminology concept codes and/or by extension, standardized medical concept codes. In some embodiments, the collection of interface terminology concept codes can include more concept codes than any of the standardized concept code sources (e.g., more interface terminology concept codes than the number of concept codes in ICD-10). Having a greater number of interface terminology concept codes can provide greater granularity in describing medical concepts as compared to one or more of the standardized medical sources. The concept analysis application 128 can search and/or filter the interface terminology concept codes based on hierarchical information and/or other description data associated with each of the concept codes. In some embodiments, the concept analysis application 128 can be used to find standardized concept codes (e.g., ICD-10 codes), find relevant patient cases for a given condition, symptoms, patient demographic, and/or other parameters, and/or lookup other suitable medical information.

In some embodiments, the concept analysis application 128 can generate one or more reports based on search results and/or otherwise provide a graphical user interface (GUI) to facilitate lookup of medical concept codes and/or patient cases.

As shown in FIG. 1, the communication network 112 can facilitate communication between the computing device 104, the supplemental computing device 116, the medical terminology source(s) 120, and the concept mapping database 124. In some embodiments, the communication network 112 can be any suitable communication network or combination of communication networks. For example, the communication network 112 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), a wired network, etc. In some embodiments, the communication network 112 can be a local area network, a wide area network, a public network (e.g., the Internet), a private or semi-private network (e.g., a corporate or university intranet), any other suitable type of network, or any suitable combination of networks. Communications links shown in FIG. 1 can each be any suitable communications link or combination of communications links, such as wired links, fiber optic links, Wi-Fi links, Bluetooth links, cellular links, and the like.

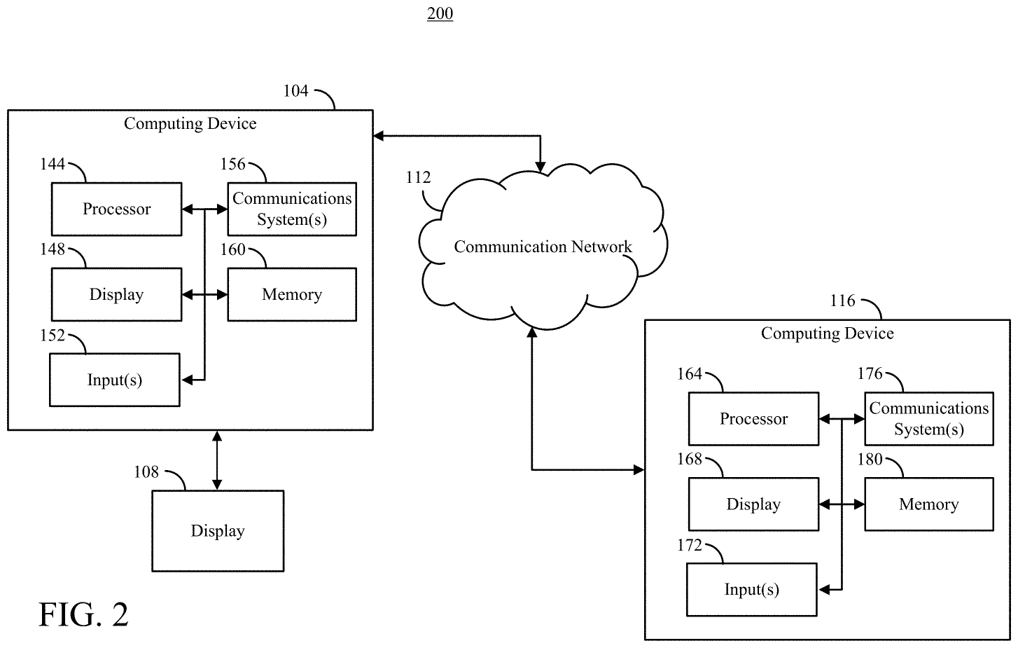

FIG. 2 shows an example 200 of hardware that can be used to implement a computing device 104 and a supplemental computing device 116 shown in FIG. 1 in accordance with some aspects of the disclosed subject matter. As shown in FIG. 2, the computing device 104 can include a processor 144, a display 148, an input 152, a communication system 156, and a memory 160. The processor 144 can implement at least a portion of the concept analysis application 128, which can, for example, be executed from a program (e.g., saved and retrieved from the memory 160). The processor 144 can be any suitable hardware processor or combination of processors, such as a central processing unit (“CPU”), a graphics processing unit (“GPU”), etc., which can execute a program, which can include the processes described below. 100341 In some embodiments, the display 148 can present a graphical user interface. In some embodiments, the display 148 can be implemented using any suitable display devices, such as a computer monitor, a touchscreen, a television, etc. In some embodiments, the inputs 152 of the computing device 104 can include indicators, sensors, actuatable buttons, a keyboard, a mouse, a graphical user interface, a touch-screen display, etc. In some embodiments, the inputs 152 can allow a user (e.g., a medical practitioner, such as an oncologist) to interact with the computing device 104, and thereby to interact with the supplemental computing device 116 (e.g., via the communication network 112). The display 108 can be a display device such as a computer monitor, a touchscreen, a television, and the like.

In some embodiments, the communication system 156 can include any suitable hardware, firmware, and/or software for communicating with the other systems, over any suitable communication networks. For example, the communication system 156 can include one or more transceivers, one or more communication chips and/or chip sets, etc. In a more particular example, the communication system 156 can include hardware, firmware, and/or software that can be used to establish a coaxial connection, a fiber optic connection, an Ethernet connection, a USB connection, a Wi-Fi connection, a Bluetooth connection, a cellular connection, etc. In some embodiments, the communication system 156 allows the computing device 104 to communicate with the supplemental computing device 116 (e.g., directly, or indirectly such as via the communication network 112).

In some embodiments, the memory 160 can include any suitable storage device or devices that can be used to store instructions, values, etc., that can be used, for example, by the processor 144 to present content using the display 148 and/or the display 108, to communicate with the supplemental computing device 116 via communications system(s) 156, etc. The memory 160 can include any suitable volatile memory, non-volatile memory, storage, or any suitable combination thereof. For example, the memory 160 can include RAM, ROM, EEPROM, one or more flash drives, one or more hard disks, one or more solid state drives, one or more optical drives, etc. In some embodiments, the memory 160 can have encoded thereon a computer program for controlling operation of the computing device 104 (or the supplemental computing device 116). In such configurations, the processor 144 can execute at least a portion of the computer program to present content (e.g., user interfaces, images, graphics, tables, reports, and the like), receive content from the supplemental computing device 116, transmit information to the supplemental computing device 116, and the like.

Still referring to FIG. 2, the supplemental computing device 116 can include a processor 164, a display 168, an input 172, a communication system 176, and a memory 180. The processor 164 can implement at least a portion of the concept analysis application 128, which can, for example, be executed from a program (e.g., saved and retrieved from the memory 180). The processor 164 can be any suitable hardware processor or combination of processors, such as a central processing unit (CPU), a graphics processing unit (GPU), and the like, which can execute a program, which can include the processes described below.

In some embodiments, the display 168 can present a graphical user interface. In some embodiments, the display 168 can be implemented using any suitable display devices, such as a computer monitor, a touchscreen, a television, etc. In some embodiments, the inputs 172 of the supplemental computing device 116 can include indicators, sensors, actuatable buttons, a keyboard, a mouse, a graphical user interface, a touch-screen display, etc. In some embodiments, the inputs 172 can allow a user (e.g., a medical practitioner, such as an oncologist) to interact with the supplemental computing device 116, and thereby to interact with the computing device 104 (e.g., via the communication network 112).

In some embodiments, the communication system 176 can include any suitable hardware, firmware, and/or software for communicating with the other systems, over any suitable communication networks. For example, the communication system 176 can include one or more transceivers, one or more communication chips and/or chip sets, etc. In a more particular example, the communication system 176 can include hardware, firmware, and/or software that can be used to establish a coaxial connection, a fiber optic connection, an Ethernet connection, a USB connection, a Wi-Fi connection, a Bluetooth connection, a cellular connection, and the like. In some embodiments, the communication system 176 allows the supplemental computing device 116 to communicate with the computing device 104 (e.g., directly, or indirectly such as via the communication network 112).

In some embodiments, the memory 180 can include any suitable storage device or devices that can be used to store instructions, values, and the like, that can be used, for example, by the processor 164 to present content using the display 168 and/or the display 108, to communicate with the computing device 104 via communications system(s) 176, and the like. The memory 180 can include any suitable volatile memory, non-volatile memory, storage, or any suitable combination thereof. For example, the memory 180 can include RAM, ROM, EEPROM, one or more flash drives, one or more hard disks, one or more solid state drives, one or more optical drives, etc. In some embodiments, the memory 180 can have encoded thereon a computer program for controlling operation of the supplemental computing device 116 (or the computing device 104). In such configurations, the processor 164 can execute at least a portion of the computer program to present content (e.g., user interfaces, images, graphics, tables, reports, and the like), receive content from the computing device 104, transmit information to the computing device 104, and the like.

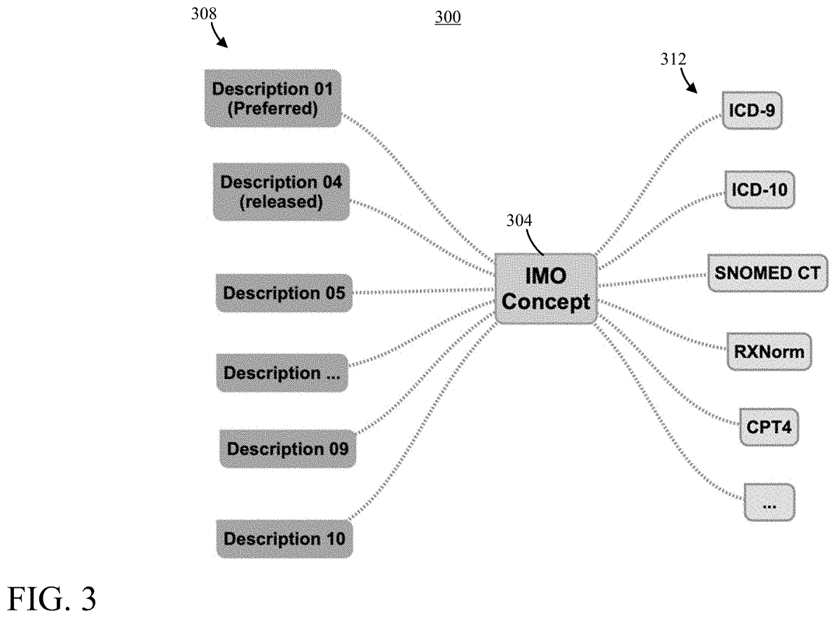

FIG. 3 shows an example of a simple interface terminology concept mapping 300. An interface terminology concept 304 can be associated with a number of descriptions 308 and a number of standardized concept codes 312. Descriptions can be alternative or semantically different ways to express the concept. In some embodiments, the standardized concept codes 312 can include, e.g., ICD-9, ICD-10 (e.g., ICD-10-CM, ICD-10-UK, etc.), SNOMED CT (i.e., SCT), RXNorm, and/or CPT4 concept codes. Thus, the interface terminology concept code 304 can have equivalent concept codes in a number of standardized concept mappings. The descriptions 308 can provide descriptor words and/or phrases associated with the interface terminology concept 304, but may not provide information about how the interface terminology concept 304 is related to other interface terminology concepts. In other words, the simple interface terminology concept mapping 300 may lack hierarchical mapping.

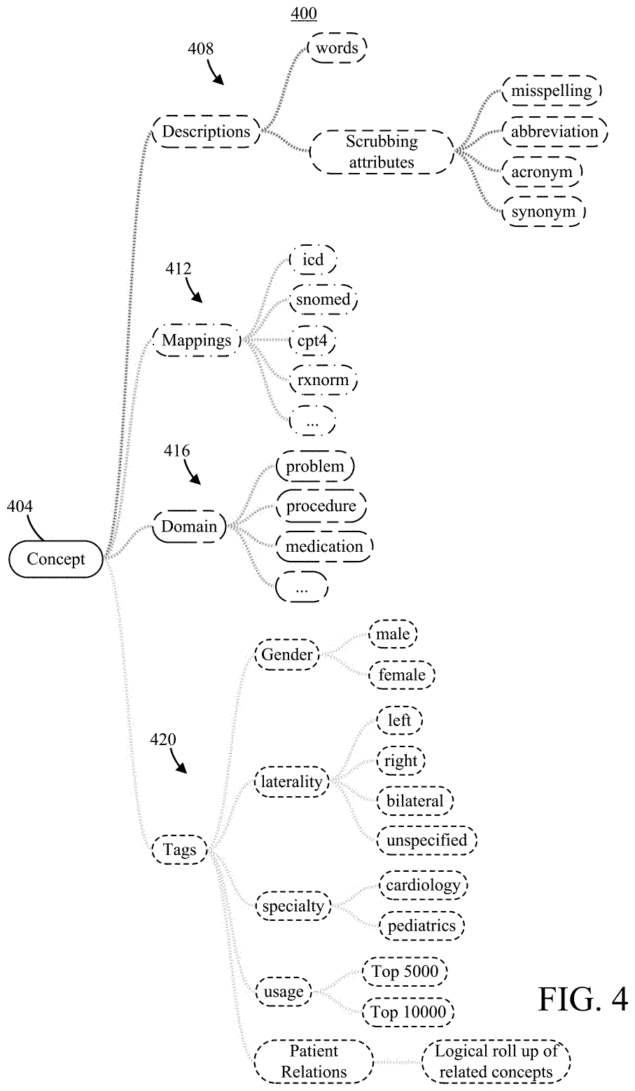

Referring now to FIG. 4, a more detailed explanation or mapping 400 of the left-hand portion of the mapping 300 of FIG. 3 is shown. In this mapping 400, an interface terminology concept code 404 can be associated with a number of descriptions 408 and a number of standardized concept codes 412. In some embodiments, for each concept, the number of descriptions 408 and the number of standardized concept codes 412 can be the same as the number of descriptions 308 and a number of standardized concept codes 312 in FIG. 3. As shown, in addition to being alternative ways to express a concept, the descriptions 408 also may include classification attributes that can account for misspellings, abbreviations, acronyms, and/or synonyms of words or phrases in the interface terminology concept 404.

Additionally, the more detailed mapping 400 can include a domain 416 and/or a number of tags 420. In this regard, each concept may be unique within a given domain. The domain 416 can provide hierarchical information about a broad and/or narrow field the concept 404 falls into. In some embodiments, the domain 416 can be a problem the interface terminology concept 404 embodies and/or includes. In some embodiments, the domain 416 can be a procedure the interface terminology concept 404 embodies and/or includes. In some embodiments, the domain 416 can be a medication the interface terminology concept 404 embodies and/or includes. In some embodiments, the concept 404 can be mapped to a single domain.

The number of tags 420 can provide granular information about specific instances of the interface terminology concept 404 without losing or diluting the overall meaning of the interface terminology concept 404. In some embodiments, the number of tags 420 can include a gender tag, a laterality tag, a specialty tag, a usage tag, and/or a relationship tag. The gender tag can indicate if the concept is related to a specific gender (e.g., female, male), or is not gender specific. For example, if the interface terminology concept 404 is “prostate cancer,” the gender tag can indicate that the concept is related to males. The laterality tag can indicate a laterality of the interface terminology concept 404 (e.g., if the interface terminology concept 404 is “ACL tear in left knee,” the laterality tag can indicate a left ACL tear). The usage tag can indicate how common and/or popular the interface terminology concept 404 is in a collection of medical records. The usage tag can be used to filter out less common interface terminology concept codes during searches, which may speed up the search process for a user. The specialty tag can indicate a specialty of a medical practitioner (e.g., a medical doctor) who generated a medical record. The relationship tag can indicate a number of related interface terminology concept codes. In some embodiments, related interface terminology concept codes can be associated with patients also associated with the interface terminology concept 404. For example, if the interface terminology concept 404 is “pneumonia,” the relationship tag can indicate a “cough” interface terminology concept. As will be described below, the relationship tag can be used to generate graphs and/or other visual indicators to show a user related concepts. In some embodiments, the visual indicators can include an associated concepts portion, which can display the most commonly related concepts, with more commonly related concepts displayed as larger than less commonly related concepts (e.g., larger boxes for more commonly related concepts). In some embodiments, the number of tags 420 can include an age tag.

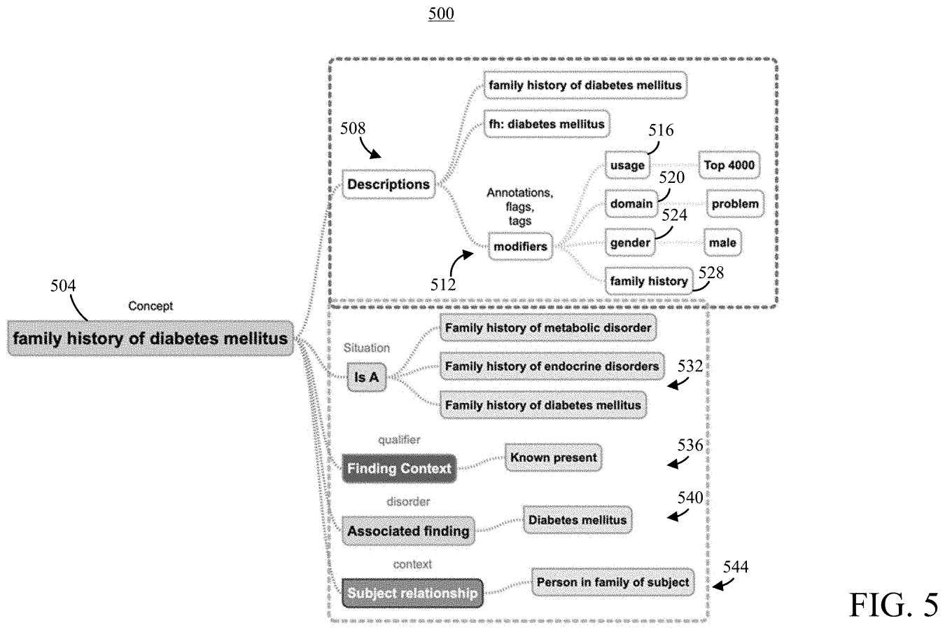

Referring now to FIG. 5, an improved interface terminology concept code mapping 500 is shown, in which semantic tagging is added to the mapping of FIG. 4. In the interface terminology concept mapping 500, an interface terminology concept code 504 can be “family history of diabetes mellitus.” The interface terminology concept code 504 can be associated with a number of descriptions 508 including the full interface terminology concept code (e.g., “family history of diabetes mellitus”), an alternate description (e.g., an abbreviated description “fh: diabetes mellitus”), and a number of modifiers 512. The modifiers 512 can include a number of annotations, flags, tags, and/or other suitable modifiers that may aid a user in searching for relevant interface terminology concept codes. The modifiers 512 can include a usage modifier 516 (e.g., “Top 4000”), a domain modifier 520 (e.g., “problem”), a gender modifier 524 (“male”), and/or a family history modifier 528, although not all modifiers may be applicable. In particular, the semantic metatags (e.g., 532, 536, 540, 544) may be derived from one or more of the public concept code ontologies to which concepts of the interface terminology are mapped. In particular, one or more of the semantic metatags may be derived from a SNOMED CT or a LOINC taxonomy. For example, the SNOMED CT ontology may include semantic tagging (e.g., “finding,” “disorder,” “event,” etc.) attached to each of its concepts that may be used as a foundation for semantic metatagging of the interface terminology concepts. In some embodiments, one or more of the semantic metatags may not be derived from a public taxonomy. In some embodiments, the interface concept code 504 can include (i.e., inherently include) information about the family history of diabetes mellitus being associated with the gene X. In this example, the association of gene X with the family history of diabetes concept may not be present in public ontologies. In another example, if the interface terminology concept code 504 is “carrier of gene X,” SNOMED CT or other public ontologies may not include a direct semantic map, and the semantic metatags 532, 536, 540, 544 can be generated based on private information. In some embodiments, the semantic metatags can be system-derived and/or user-derived.

In order to provide granularity to interface terminology concepts, a number of intermediate tables can be generated based on public concept codes (e.g., SNOMED CT concepts). In some embodiments, a public concept code can be a publicly available concept code and/or a concept code included in an external ontology (e.g., an ontology other than the interface terminology concepts ontology). In some embodiments, a process can include generating a flattened table of interface terminology concepts, generating a flattened table of public concept codes, determining a relationship map between the public concept codes (e.g., a hierarchical map), generating a relationship table including relationships between the public concept codes based on the relationship map, harvesting a number of attributes from the interface terminology concepts, and mapping each public concept code to at least one interface terminology concept based on the harvested attributes and the relationship map. Because the interface terminology may be more granular than each of the public concept ontologies, more than one interface terminology concept may map to a given public concept code, e.g., a SNOMED CT code. Additionally, each interface terminology concept may map to more than one public concept code.

In one aspect, the semantic metatags may be derived from a hybridized model, in which the container, e.g., “Is a,” “Finding Context,” “Associated Finding,” and “Subject Relationship” in FIG. 5 are derived from one or more of the public concept ontologies, while the values within each container are derived from the interface terminology mapping. Each container may have more than one suitable descriptor that is included in a public concept ontology. Each value in the container can be a single “unifying” value that is used consistently in the interface terminology concept mapping 500. For example, there may be many different values that describe “known present,” but only “known present” may be used for suitable “Finding Context” containers.

In another aspect, the semantic metatags may be derived using one or more machine learning techniques. In some embodiments, a classifying technique (e.g., an unsupervised learning technique) such as k nearest neighbors (KNN) (i.e., k-means clustering) can be used to group outputs from a natural language processor (NLP). For example, an NLP can extract information from patient records, doctor notes, and/or other records, and output a number of “meanings” based on the extracted information. A KNN technique can then be used to generate a number of semantic metatags based on the meanings and/or relationships between the meanings. In this way, interface terminology concept codes associated with similar meanings generated by the NLP can be grouped together. In some embodiments, the NLP can be the Bidirectional Encoder Representations from Transformers (BERT) developed by Google. In some embodiments, a supervised machine learning technique can train a model to map additional interface terminology concept codes based on an existing interface terminology concept code structure.

In some embodiments, an existing general lexical database (e.g., WordNet) can be used to generate semantic metatags. For example, a KNN technique can be used to group similar medical terms in the lexical database into similar clusters. For example, medical terms with shared synonyms can be clustered together. In some embodiments, the NLP can be the Bidirectional Encoder Representations from Transformers (BERT) developed by Google.

As seen in FIG. 5, the interface terminology is structured with the additional mapping attributes mapping to each concept, whereas the other modifiers discussed above are mapped to respective descriptions in the ontology. The descriptions 508 can capture common aspects of interface terminology concept codes. The semantic metatags can add “fine grained” metadata to the interface terminology concept code 504. In some embodiments, the semantic metatags may not be visible to an outside user (e.g., the semantic metatags can be visible to backend users and/or processes only). The semantic metatags can improve the performance of search and/or matching processes, as well as improve the robustness of mapping in general.

The interface terminology concept code 504 can be associated with a situation attribute 532. The situation attribute can be generated and/or pulled from a public terminology (e.g., SNOMED CT). The situation attribute 532 can include one or more categories populated with one or more values. As shown, the situation attribute 532 can include an “Is A” category populated, e.g., with “Family history of metabolic disorder,” “Family history of endocrine disorders,” and “Family history of diabetes mellitus” values. Notably, the situation attribute 532 can include a number of values that differ from the interface terminology concept code 504. In particular, the situation attribute 532 can include a number of values that are broader than (e.g., are parents to) and/or that are narrower than (e.g., are children to) the interface terminology concept code 504. For example, “Family history of metabolic disorder” and “Family history of endocrine disorders” are broader than, and can be parents to, the “Family history of diabetes mellitus” value. In some aspects, the values may be referred to as situation attributes.

The interface terminology concept code 504 can be associated with a qualifier attribute 536. The qualifier attribute 536 can include one or more categories populated with one or more values. As shown, the qualifier attribute 536 can include a “Finding Context” category populated with a “Known present” value, which can indicate the status of the concept is currently known. In some aspects, the values may be referred to as qualifier attributes.

The interface terminology concept code 504 also may be associated with a disorder attribute 540. The disorder attribute 540 can include one or more categories populated with one or more values. As shown, the disorder attribute 540 can include an “Associated finding” category populated with a “Diabetes mellitus” value, which can indicate the associated disorder of the interface terminology concept code 504 is diabetes mellitus. In some aspects, the values may be referred to as disorder attributes.

The interface terminology concept code 504 can be associated with a context attribute 544. The context attribute 544 can include one or more categories populated with one or more values. As shown, the context attribute 544 can include a “Subject relationship” category populated with a “Person in family of subject” value, which can indicate that person having the disorder associated with the interface terminology concept code 504 (e.g., diabetes mellitus) is a family member of the patient. In some aspects, the values may be referred to as context attributes.

In some embodiments, the situation attribute 532 values, the qualifier attribute 536, the disorder attribute 540, and/or the context attribute 544 can be generated using an attribute generation process as detailed below.

In some embodiments, an attribute generation process can generate the situation attribute 532, the qualifier attribute 536, the disorder attribute 540, and/or the context attribute 544. In some embodiments, the attribute generation process can include determining if a concept code contains one or more predetermined substrings, determining a number of attributes based on the substrings included in the concept code, and assigning the number of attributes to the concept code. The predetermined substrings can be previously generated based on public concept codes (e.g., SNOMED CT concept codes).

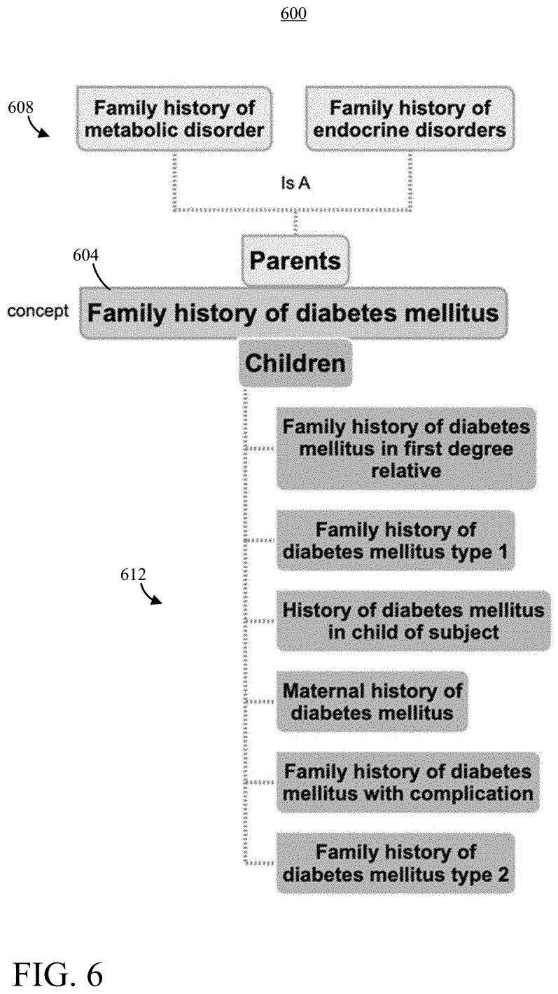

Referring now to FIG. 6, an exemplary ontological graph 600 of an interface terminology concept code 604 is shown. The interface terminology concept code 604 can be associated with a number of parent concept codes 608 and a number of child concept codes 612. In some embodiments, the parent concept codes 608 and the child concept codes 612 can be interface terminology concept codes. The parent concept codes 608 can be broader than the interface terminology concept code 604, and the child concept codes 612 can be narrower than the interface terminology concept code 604. Thus, the ontological graph 600 can include a hierarchical ordering of the interface terminology concept code 604, the parent concept codes 608, and the number of child concept codes 612. In some embodiments, a user can traverse the hierarchical ordering using the concept analysis application 128.

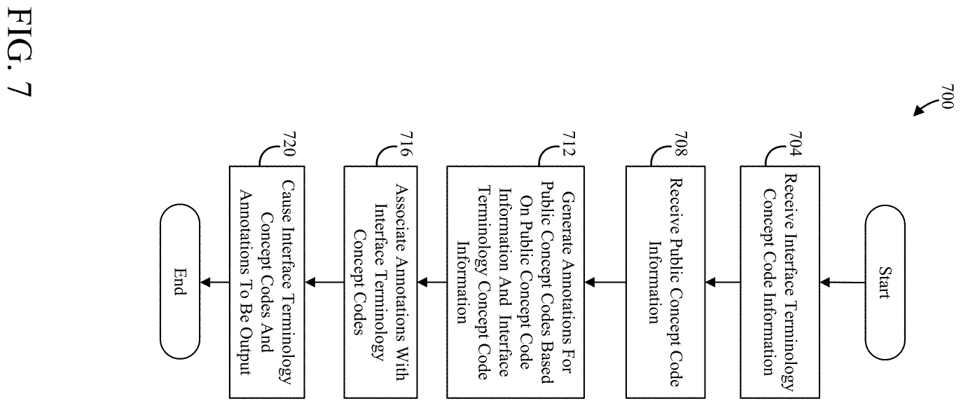

Referring now to FIG. 7, an exemplary process 700 for generating annotations for interface terminology concept codes based on public concept codes is shown. In some embodiments, the process 700 can be implemented as computer readable instructions on a non-transitory computer readable medium such as a memory (e.g., the memory 160 and or the memory 180 in FIG. 2) and executed by a processor (e.g., the processor 144 and/or the processor 164 in FIG. 2).

At 704, the process 700 can receive interface terminology concept code information. The interface terminology concept code information can include a number of interface terminology concept codes, a number of public concept codes associated with a predetermined medical terminology (e.g., SNOMED CT), and a number of descriptions, each interface terminology concept code being associated with one of the public concept codes and one of the descriptions. The interface terminology concept code information can also include a map of the number of interface terminology concept codes. For example, the map can include the interface terminology concept codes as nodes included in the map and connected by a number of edges.

At 708, the process 700 can receive public concept code information. The public concept code information can include a number of concept codes, each concept code associated with a description, an active indicator, a source ID, a destination ID, a relationship group, and/or a modifier ID. The public concept code information can be associated with a predetermined medical terminology (e.g., SNOMED CT).

At 712, the process 700 can generate annotations for the public concept codes based on the public concept code information and the interface terminology concept code information. In some embodiments, the process 700 can generate one or more annotations by determining a string and/or substring of the description associated with each public concept code contains an annotation that align with one or more predetermined annotation categories. In some embodiments, the process 700 can “overlay” annotations on each public concept code, and add granularity to a public concept code. In some embodiments, the process 700 can generate the annotations and associate but not overlay the annotations on a concept code. In this way, the concept codes can remain the same, but searching and mapping of the public concept codes can be improved because the associated annotations can add supplemental granularity. In some embodiments, the annotation categories can include finding, disorder, event, procedure, qualifier, body structure, substance, situation, context, attribute, morphology, observable entity, disposition, organism, and/or specimen. The process 700 can search the description associated with the public concept code for one or more annotations (e.g., values) that fall into at least one of the annotation categories. The process 700 can include receiving a predetermined list of values corresponding to the annotation categories. In some embodiments, the process 700 can generate the annotations using one or more machine learning techniques. In some embodiments, the process 700 can map the public concept codes to the interface terminology concept codes (e.g., generate new nodes and/or edges in the map) using a model trained using a supervised machine learning technique.

At 716, the process 700 can associate the annotations with the interface terminology concept codes. The process 700 can include assigning the annotation categories and the annotations generated for each public concept code to each interface terminology concept code associated with the public concept code. In some embodiments, the annotations can be used to search and/or filter interface terminology concept codes.

At 720, the process 700 can cause the interface terminology concept codes and the associated annotations to be output. In some embodiments, the process 700 can cause the interface terminology concept codes and the associated annotations to be output to a database such as the concept mapping database 124 in FIG. 1. The public concept codes and/or the associated annotations can then be used directly as descriptions, modifiers, situations, qualifiers, contexts, disorders, domains, standardized concept codes, etc., as described above in conjunction with FIGS. 4 and 5. In some embodiments, the annotations can be used to indirectly generate the descriptions, modifiers, situations, qualifiers, contexts, disorders, domains, standardized concept codes, etc.

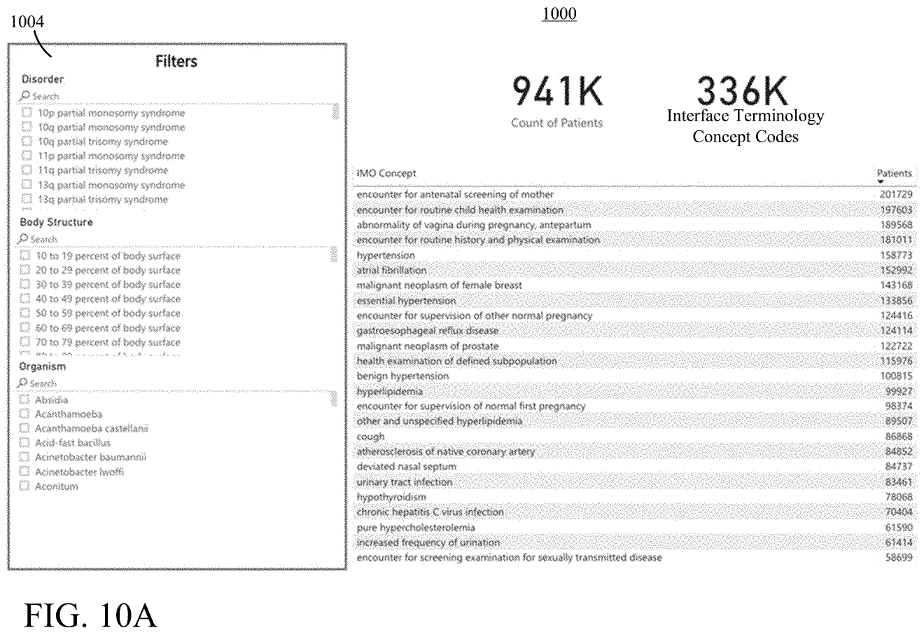

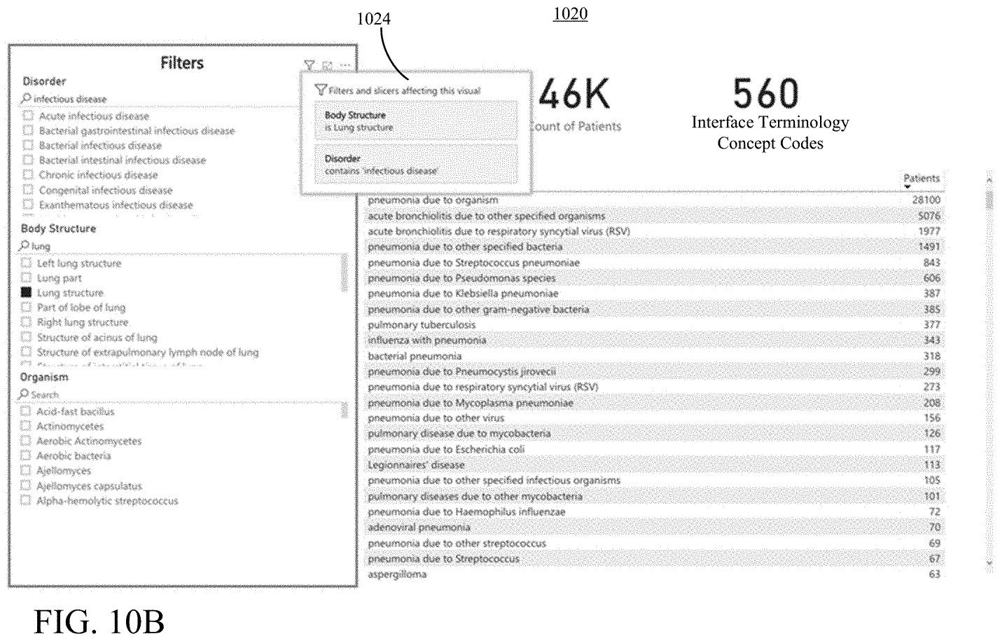

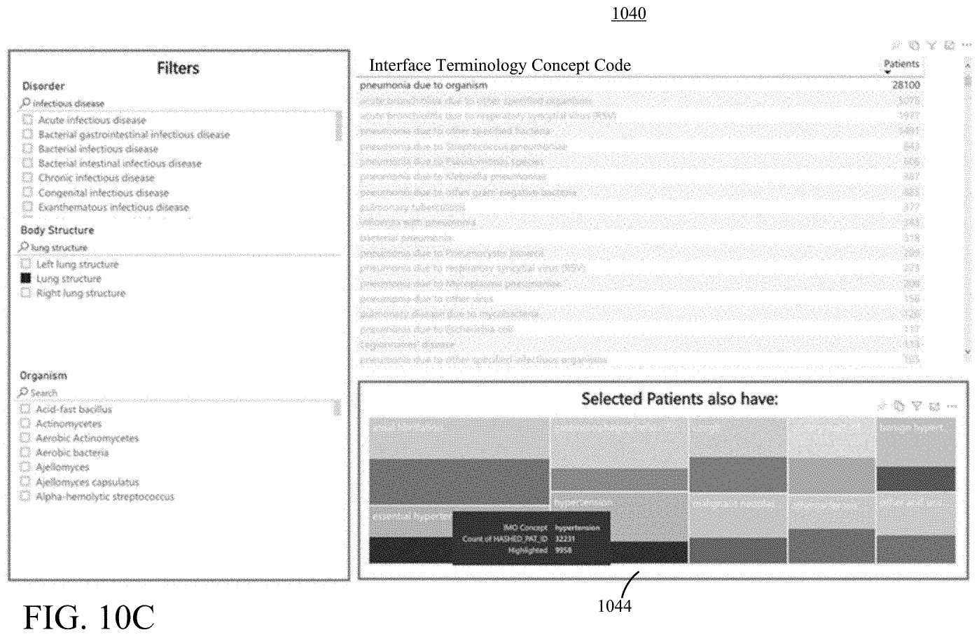

Once mapped to the public concept codes and/or the associated annotations, the interface terminology concept codes can be referred to as mapped interface terminology concept codes. The mapped interface terminology concept codes can be used for a number of different applications. In some embodiments, the mapped interface terminology concept codes can be used to improve search processes and/or query processes for patient medical records, clinical reports, and/or other suitable information that can be tagged with the interface terminology concept codes. For example, in a database of patient medical records, each medical record can be associated with at least one mapped interface terminology concept code. As described above, the annotations for each interface terminology concept code can be associated with descriptions, modifiers, situations, qualifiers, contexts, disorders, domains, standardized concept codes directly or indirectly generated from the annotations. In some embodiments, the concept analysis application 128 can receive a search query including at least one word, and determine which interface terminology concept codes are most relevant to the search query by comparing the search query to the annotations. The semantic metatagging described above may improve the client query process in several ways. For example, each semantic metatag may represent an additional query option for determining concepts that did not exist previously, thereby providing the user with improved techniques for identifying potentially relevant concepts beyond identifying them as a result of matches to concept or description keyword queries. Additionally, the semantic metatagging may cause interface terminology concepts to become related in previously non-existent ways. For example, concepts within the interface terminology previously may have been hierarchically related — either through the interface terminology concepts themselves being part of a hierarchy or through a hierarchy from one of the public concept codes (e.g., a SNOMED CT hierarchy) being applied via the external mappings to the interface terminology concepts. The semantic metatagging described herein may cause interface terminology concepts outside of those hierarchical relationships to now be related due to their semantic similarities. For example, the concepts “acute bronchiolitis due to respiratory syncytial virus (RSV)” may be semantically related to “pulmonary tuberculosis” due to both concepts being tagged with a “body structure” container value of “lung structure” and a “disorder” container value of “contains ‘infectious disease,’” even though the two concepts are not hierarchically related (see FIG. 10B).

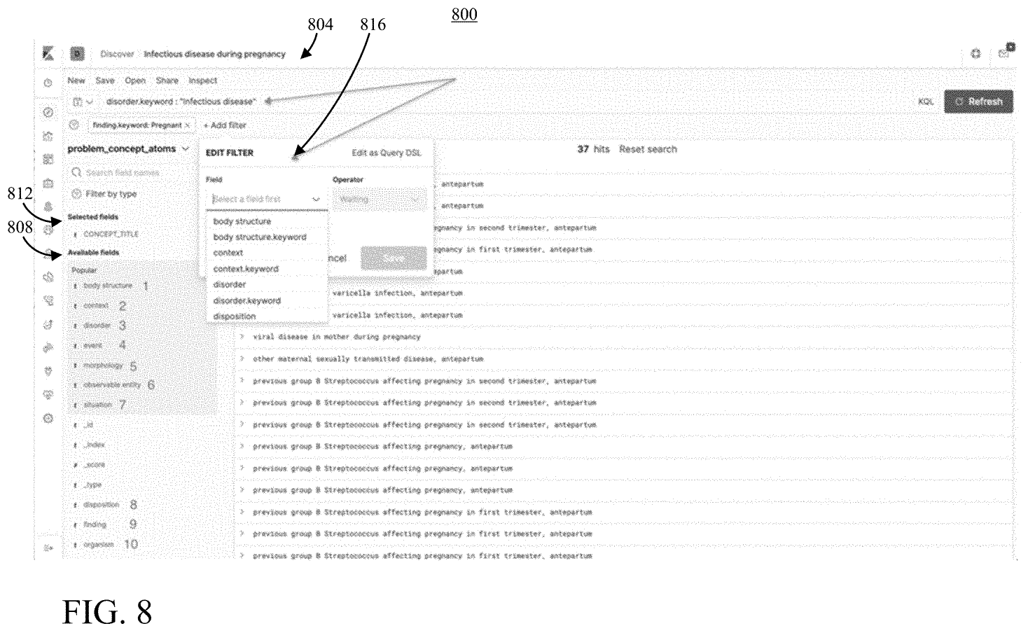

Referring now to FIG. 8, an exemplary GUI 800 for searching a number of interface terminology concept codes is shown. The GUI can be included in the concept analysis application 128. In some embodiments, the GUI 800 can include a search bar 804, an available fields portion 808, a selected fields portion 812, and a filter editing portion 816. The search bar 804 can receive input (e.g., a string of words) from a user. The available fields portion 808 can include a number of fields such as attributes that can be selected by a user to filter search results. The selected fields portion 812 can include a number of fields that have already been selected. The filter editing portion 816 can receive input from a user in order to generate filters and narrow the search results. In some embodiments, the filter editing portion 816 can receive a field selection from a user and an operator associated with the field selection, and filter the results to only present interface terminology concept codes related to the operator. In some embodiments, the field selection can be an annotation category such as finding, disorder, event, procedure, qualifier, body structure, substance, situation, context, attribute, morphology, observable entity, disposition, organism, and/or specimen. In such embodiments, the operator can be a value selected from a predetermined list corresponding to the annotation category. For example, the field selection can be “situation,” and the operator can be “family history of endocrine disorder.” As another example, the field selection can be “qualifier,” and the operator can be “current or past.”

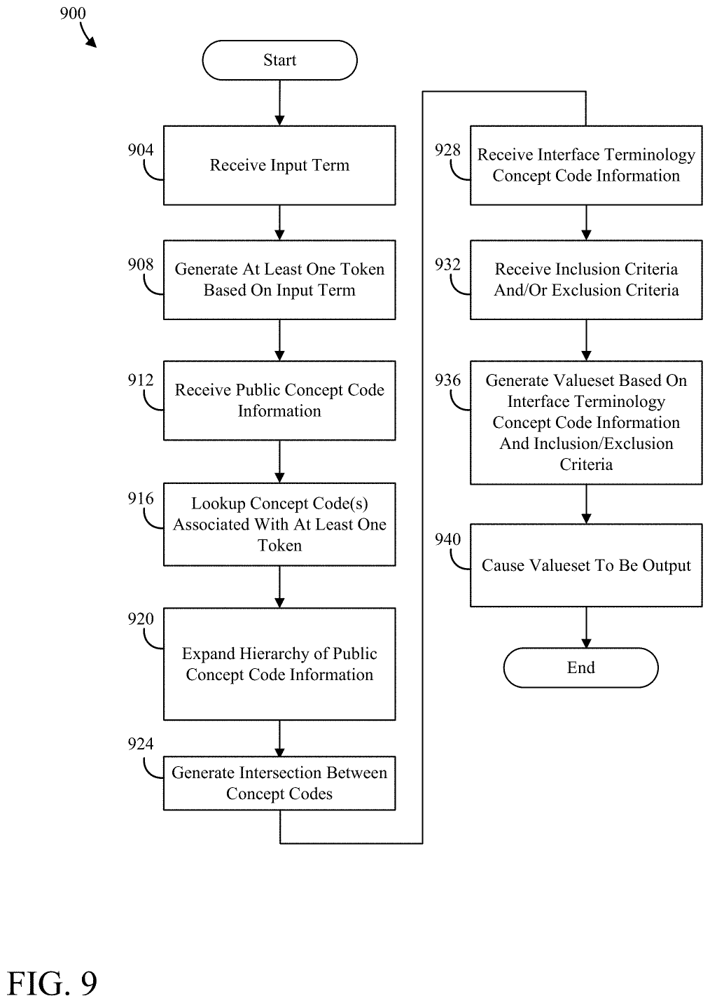

Referring now to FIG. 9, an exemplary process 900 for generating valuesets representing hierarchical relationships between concepts is shown. In some embodiments, the process 900 can be implemented as computer readable instructions on a non-transitory computer readable medium such as a memory (e.g., the memory 160 and or the memory 180 in FIG. 2) and executed by a processor (e.g., the processor 144 and/or the processor 164 in FIG. 2).

At 904, the process 900 can receive an input term. The input term can include a number of words. For example, the input term can be “backache with sciatica.”

At 908, the process 900 can generate at least one token based on the input term. In some embodiments, the tokens can include values corresponding to categories including finding, disorder, event, procedure, qualifier, body structure, substance, situation, context, attribute, morphology, observable entity, disposition, organism, and/or specimen. For example, if the input term is “backache with sciatica,” the process 900 can generate a finding token populated with a “backache” value and a disorder token populated with a “sciatica” value.

At 912, the process 900 can receive public concept code information. In some embodiments, the public concept code information can include a public concept code and all hierarchically related concept codes (e.g., parent and/or children concept codes). In this way, the public concept code information can include a hierarchy of public concept codes.

At 916, the process 900 can lookup public concept code information for the at least one token. For example, the process 900 can lookup information for the finding token populated with a “backache” value and the disorder token populated with a “sciatica” value.

At 920, the process 900 can expand the hierarchy of public concept codes. In some embodiments, the process 900 can flatten the hierarchy by grouping the public concept code and the associated parent concept codes and/or children concept codes in a single group. In some embodiments, the process 900 can group the public concept code and the associated parent concept codes and/or children concept codes based on non-hierarchical data such as descriptions, modifiers, situations, qualifiers, contexts, disorders, etc.

At 924, the process 900 can generate an intersection between concept codes associated with the at least one token.

At 928, the process 900 can receive interface terminology concept code information. The interface terminology concept code information can include a number of interface terminology concept codes associated with the public concept codes. In some embodiments, each of the interface terminology concept codes can be linked to one or more of the public concept codes. Each of the interface terminology concepts codes can be associated with a number of attributes and/or operators as described above.

At 932, the process 900 can receive inclusion criteria and/or exclusion criteria. In some embodiments, the inclusion criteria can include a number of keywords, attributes, and/or operators used to keep relevant interface terminology concept codes in a valueset. In some embodiments, the exclusion criteria can include a number of keywords, attributes, and/or operators used to filter irrelevant interface terminology concept codes out of the valueset.

At 936, the process 900 can generate the valueset based on the interface terminology concept code information and the inclusion criteria and/or exclusion criteria. The process 900 may only include interface terminology concept codes that include the inclusion criteria and not include the exclusion criteria in the valueset. In some embodiments, the valueset can be extensional. An extensional valueset can include the interface terminology concept codes and/or public interface terminology codes. In some embodiments, the valueset can be intensional. An intensional valueset can include a rule based on semantic tags. The rule can be used to generate a list of interface terminology concept codes and/or public interface terminology codes included in an extensional valueset.

At 940, the process 900 can cause the valueset to be output. In some embodiments, the process 900 can cause the valueset to be output to a database that includes patient data linked by the interface terminology concept codes such as the concept mapping database 124 in FIG. 1. Patients included in a database can individually be associated with one or more interface terminology concept codes and/or public interface terminology codes. In some embodiments, a filter can identify patients matching one or more interface terminology concept codes and/or public interface terminology codes included in a valueset. In this way, a search engine for the database can identify relevant patients (e.g., for a user) based on one or more valuesets. In some embodiments, the search engine can filter patients (e.g., with a filter) by identifying patients in the database that have some combination of the interface terminology concept codes and/or public interface terminology codes included in one or more valuesets. In some embodiments, a filter can be generated based on multiple valuesets by combining all interface terminology concept codes and/or public interface terminology codes included in a plurality of valuesets. The filter may act as a “cohort.” In some embodiments, a filter can be generated dynamically. For example, a filter can be generated based on an extensional valueset generated based on a rule included in an intensional valueset. As another example, the filter can be generated in real-time based on input from the user.

Referring now to FIG. 10A, an exemplary GUI 1000 for searching a patient database using a number of interface terminology concept codes is shown. More specifically, the GUI 1000 can be used (e.g., by a user) to filter patients by concept codes (e.g., interface terminology concept codes and/or public interface terminology codes) using one or more valuesets as described above. The GUI 1000 can be included in the concept analysis application 128. In some embodiments, the GUI 1000 can include a first portion of a display that includes a filter editing portion 1004. The filter editing portion 1004 can receive input from a user in order to generate filters and narrow the search results. In some embodiments, the filter editing portion 1004 can receive an operator associated with a field selection from a user, and filter the results to only present interface terminology concept codes related to the operator. In some embodiments, the filter editing portion 1004 can select a previously generated valueset based on input from a user, and filter patients matching concept codes included in the valueset. In some embodiments, the filter editing portion 1004 can generate a new filter based on one or more valuesets selected based on the input from the user. A second portion of the interface may be responsive to the user selections in the first portion, presenting and then modifying a display of interface terminology concepts based on inclusion and/or exclusion criteria chosen by the user. In some embodiments, the criteria can be including or excluding a list of concept codes (e.g., concept codes included in a valueset) or including or excluding a code and subsumed descendants of the codes, (e.g., all children of the code). In some embodiments, the field selection can be an annotation category such as finding, disorder, event, procedure, qualifier, body structure, substance, situation, context, attribute, morphology, observable entity, disposition, organism, and/or specimen. In such embodiments, the operator can be a value selected from a predetermined list corresponding to the annotation category. For example, the field selection can be “organism,” and the operator can be “absidia.” As another example, the field selection can be “body structure,” and the operator can be “10 to 19 percent of body surface.”

Referring now to FIG. 10B, another exemplary GUI 1020 for searching a patient database using a number of interface terminology concept codes is shown. The GUI 1020 can be included in the concept analysis application 128. In some embodiments, the GUI 1020 can include a selected filters portion 1024 that includes selected filters. As with the GUI of FIG. 10A, the GUI of FIG. 10B may include a first portion and a second portion, where the second portion is configured to dynamically respond and update in response to user entry in the first portion. In some embodiments, the selected filters can include a field selection and an associated operator.

Referring now to FIG. 10C, yet another exemplary GUI 1040 for searching a patient database using a number of interface terminology concept codes is shown. The GUI 1040 can be included in the concept analysis application 128. In some embodiments, the GUI 1040 can include an associated concepts portion 1044 that includes common interface terminology concept codes also associated with a filtered pool of patients. In some embodiments, the associated concepts portion 1044 can include a graph or other visual data depiction of the most common interface terminology concept codes associated with the filtered pool of patients. In some embodiments, the graph or other visual data depiction can be generated based on relationship tags included in concept codes associates with the filtered pool of patients. The associated concepts portion 1044 can be generated by filtering interface terminology codes and/or associated metadata included in patient records, which can be derived from public information and/or private information. As with the GUI of FIGS. 10A and 10B, the GUI of FIG. 10C may include a first portion and a second portion, where the second portion is configured to dynamically respond and update in response to user entry in the first portion.

In some embodiments, a search engine such as the engine provided under the trademark Apache Spark (“Spark”) can be used to perform searches. Spark is advantageous over traditional searches for a number of reasons. Spark can handle clustered data in a lazy evaluation methodology so that compute nodes are called only when needed. Instead of running terminology transformation one by one, transformations are stored in Directed Acyclic Graph (DAG), and the whole graph is run in an efficient manner. Spark can also utilize parallel processing to improve runtime. Spark can also scale to process high volumes of data through optimized Hadoop Distributed File System (HDFS) based storage and by adding more worker nodes when needed.

The above systems and processes (e.g., the concept analysis system 100, the process 700 in FIG. 7, and/or the process 900 in FIG. 9) can provide several advantages over other methods for generating annotations and/or valuesets. One advantage over manual methods for generating annotations and/or valuesets is that it would be practically impossible to replicate an interface terminology list of concept code maps. For example, an interface terminology concept code map may include hundreds of thousands of nodes or millions of nodes connected by tens of millions of edges, which is not practically possible to reproduce on paper or in a user's mind. In some embodiments, the nodes includes edges that are subsumed descendants.

Furthermore, it would be practically impossible to replicate subsumed descendants of each interface terminology concept codes, which may be included in the concept code maps.

Additionally, human analysis of an interface terminology concept code map may lead to errors due to the sheer size of the interface terminology concept code map. The interface terminology concept code map may take a human hundreds of days to analyze due to the size of the map. Additionally, the process 700 and/or 900 can be applied to automatically add new concepts into an interface terminology concept code database. In some embodiments, processes can be used to generate patient risk scores (e.g., for insurance companies) based on a “complexity” of patient, which may include analyzing a large number of concepts and/or attributes associated with the patient, which may impossible and/or not practical for a human to do.

Thus, the present disclosure provides systems and methods for improved lookup of standardized medical terms (e.g., concepts, codes, etc.) and/or patient conditions and/or symptoms.

The present invention has been described in terms of one or more preferred configurations, and it should be appreciated that many equivalents, alternatives, variations, and modifications, aside from those expressly stated, are possible and within the scope of the invention.

1. A medical concept code analysis system comprising:
a medical terminology source;
at least one processor configured to access the medical terminology source and configured to:
receive interface terminology concept code information associated with a plurality of interface terminology concept codes;
receive public concept code information associated with a plurality of public concept codes;
generate annotations for the plurality of public concept codes based on the public concept code information by determining a string and/or substring of a description associated with each public concept code contains an annotation that align with one or more predetermined annotation categories;
associate the annotations with the plurality of interface terminology concept codes; and
cause the interface terminology concept codes and the annotations to be output.

2. The system of claim 1, wherein the causing the interface terminology concept codes and the annotations to be output comprises outputting the interface terminology concept codes and the annotations to a storage database.

3. The system of claim 1, wherein the annotation categories include at least one of finding, disorder, event, procedure, qualifier, body structure, substance, situation, context, attribute, morphology, observable entity, disposition, organism, or specimen.

4. The system of claim 1, wherein the generating annotations comprises searching the description associated with each public concept code for one or more values that fall into at least one of the annotation categories.

5. The system of claim 1, wherein the public concept codes are SNOMED CT codes.

6. A medical concept code analysis method in a non-transitory computer readable medium, the method comprising:
receiving interface terminology concept code information associated with a plurality of interface terminology concept codes;
receiving public concept code information associated with a plurality of public concept codes;
generating annotations for the plurality of public concept codes based on the public concept code information by determining a string and/or substring of a description associated with each public concept code contains an annotation that align with one or more predetermined annotation categories;
associating the annotations with the plurality of interface terminology concept codes; and
causing the interface terminology concept codes and the annotations to be output.

7. The method of claim 6, wherein the causing the interface terminology concept codes and the annotations to be output comprises outputting the interface terminology concept codes and the annotations to a storage database.

8. The method of claim 6, wherein the annotation categories include at least one of finding, disorder, event, procedure, qualifier, body structure, substance, situation, context, attribute, morphology, observable entity, disposition, organism, or specimen.

9. The method of claim 6, wherein the generating annotations comprises searching the description associated with each public concept code for one or more values that fall into at least one of the annotation categories.

10. The method of claim 6, wherein the public concept codes are SNOMED CT codes.

11. A medical concept code analysis system comprising:
a medical terminology source;
at least one processor configured to access the medical terminology source and configured to:
receive public concept code information associated with a plurality of public concept codes arranged in a hierarchy;
flatten the hierarchy;
receive interface terminology concept code information associated with a plurality of interface terminology concept codes, each of the plurality of interface terminology concept codes being associated with at least one of the plurality of public concept codes;
receive inclusion criteria and exclusion criteria;
generate a valueset of interface terminology concept codes based on the inclusion criteria and exclusion criteria; and
cause the valueset to be output.

12. The system of claim 11, wherein the hierarchy comprises a central public concept node having a plurality of parent public concept nodes and a plurality of child public concept nodes.

13. The system of claim 11, wherein the inclusion criteria comprises a number of keywords, attributes, and operators used to keep relevant interface terminology concept codes in a valueset.

14. The system of claim 11, wherein the exclusion criteria comprises a number of keywords, attributes, and operators used to filter irrelevant interface terminology concept codes out of the valueset.

15. The system of claim 11, wherein the valueset is output to a storage database.

16. A medical concept code analysis method in a non-transitory computer readable medium, the method comprising:
receiving public concept code information associated with a plurality of public concept codes arranged in a hierarchy;
flattening the hierarchy;
receiving interface terminology concept code information associated with a plurality of interface terminology concept codes, each of the plurality of interface terminology concept codes being associated with at least one of the plurality of public concept codes;
receiving inclusion criteria and exclusion criteria;
generating a valueset of interface terminology concept codes based on the inclusion criteria and exclusion criteria; and
causing the valueset to be output.

17. The method of claim 16, wherein the hierarchy comprises a central public concept node having a plurality of parent public concept nodes and a plurality of child public concept nodes.

18. The method of claim 16, wherein the inclusion criteria comprises a number of keywords, attributes, and operators used to keep relevant interface terminology concept codes in a valueset.

19. The method of claim 16, wherein the exclusion criteria comprises a number of keywords, attributes, and operators used to filter irrelevant interface terminology concept codes out of the valueset.

20. The method of claim 16, wherein the valueset is output to a storage database.
